(19) United States
(12) Patent Application Publication
(10) Pub. No.: US 2024/0158787 A1
(43) Pub. Date: May 16, 2024

(54) USE OF miRNA408 IN REGULATION OF CADMIUM ACCUMULATION IN CROP

(71) Applicant: ZHEJIANG UNIVERSITY, Hangzhou, Zhejiang (CN)

(72) Inventors: LIANG WU, HANGZHOU, ZHEJIANG (CN); ERKUI YUE, HANGZHOU, ZHEJIANG (CN); FUXI RONG, HANGZHOU, ZHEJIANG (CN); XIA WU, HANGZHOU, ZHEJIANG (CN)

(21) Appl. No.: 17/773,236

(22) PCT Filed: Sep. 14, 2021
(86) PCT No.: PCT/CN2021/118166

§ 371 Date: Apr. 29, 2022

(30) Foreign Application Priority Data
Dec. 31, 2020 (CN) 202011626941.8

(51) Int. Cl. C12N 15/113; C12N 15/82

(52) U.S. Cl. CPC C12N 15/113; C12N 15/8205; C12N 15/8261; C12N 2310/141

(57) ABSTRACT

The present disclosure provides use of miR408 in regulation of cadmium accumulation in crops, and belongs to the technical field of biology. The present disclosure constructs an overexpression vector of MIR408 precursor gene, and transforms MIR408 gain-of-function transgenic rice obtained from a recipient rice, on the one hand can significantly inhibit cadmium accumulation in rice, and on the other hand, have no fundamentally effect on other agronomic traits. Therefore, the present disclosure can provide an important genetic material for breeding low-cadmium rice, and can also be used for genetic transformation of other monocotyledonous crops, providing reference for other crops planted in cadmium-contaminated soil to reduce heavy metal hazards such as cadmium accumulation.

### CROSS REFERENCE TO RELATED APPLICATION(S)

This patent application claims the benefit and priority of Chinese Patent Application No. 202011626941.8 filed on Dec. 31, 2020, and entitled “USE OF miRNA408 IN REGULATION OF CADMIUM ACCUMULATION IN CROP”, the disclosure of which is incorporated by reference herein in its entirety as part of the present application.

### REFERENCE TO SEQUENCE LISTING SUBMITTED ELECTRONICALLY

The official copy of a substitute Sequence Listing is submitted electronically via EFS-Web as an ASCII formatted sequence listing with a file named “GWPCTP20220301530_sequence_listing.txt” created on Apr. 15, 2022, and having a size of 5,495 bytes and is filed concurrently with the specification. The sequence listing contained in this ASCII formatted document is part of the specification and is herein incorporated by reference in its entirety.

### TECHNICAL FIELD

The present disclosure relates to the technical field of biology, in particular to use of small RNA miR408 in cultivation of low-cadmium crops.

### BACKGROUND ART

“People is the basic of a country, bread is the staff of life”. Rice is an important food crop, and a staple food for more than half of the world's population. Breeding better and safer rice for people to eat is an important goal of today's rice breeding. Cadmium (Cd), a non-essential element for human body, which can cause chronic poisoning by long-term consumption of Cd-contaminated food. Rice is a cereal crop with the strongest absorption of Cd. In addition to Cd, heavy metals such as arsenic and lead can also contaminate approximately 20% of China's arable land. Therefore, whether a heavy metal content of edible parts of rice and other crops exceeds the standard has become one of important social issues in people's production and life.

At present, there are few reports about genes involved in absorption, transport and accumulation of Cd in rice. Using rice cadmium-resistant and cadmium-sensitive materials to construct a mapping population, it was found that OsHMA3, encoding a heavy-metal ATPase (P1B) family transporter, can fix Cd into root cell vacuoles, thereby reducing Cd transport from roots to stems, leaves, grains, and other aerial parts; OsNRAMP5 is a cotransporter of manganese (Mn) and Cd in rice. Although rice plants with silenced expression of OsNRAMP3 gene can significantly reduce plant Cd accumulation, Mn absorption in rice will also be hindered, therefore influencing rice growth and development and reducing rice yield; the low-affinity cation transporter OsLCT1 plays an important role in Cd transport process in rice, which is abundantly expressed in vascular bundles of rice leaves and stems. Transgenic plants with silenced expression of OsLCT1 gene do not affect Cd transport in the xylem, but significantly affect transport of Cd, so the accumulation of Cd in brown rice can be effectively reduced. Since other agronomic traits and metal ion metabolism have not been found to be regulated by OsLCT1, this gene is considered to be useful for low-Cd rice breeding.

MicroRNA (miRNA) is an important class of small RNA in plants, which plays an important role in plant growth and development, resisting external biological and abiotic stresses. miR408 is a class of plant conserved miRNA, which is processed from MIR408 precursor gene. Studies have shown that the sequences of mature miR408 in different plants are highly similar, and due to 5′end of which is not uracil (U), miR408 is selected to enter AGO2 rather than AGO1 RISCs. Target genes of plant miR408 are also quite conserved, all encoding Blue Copper family proteins. In different plants, miR408 regulates different members of subfamily proteins of Blue Copper family protein, such as laccase, phytocyanin and plastocyanin. Although the plant miR408 has been reported to be involved in a response to oxidative stresses such as low temperature and salinity, it is still unknown whether it mediates biological functions of absorption, transport and accumulation of heavy metals such as Cd, and it is unclear whether it can be used in low-cadmium rice breeding.

### SUMMARY

An objective of the present disclosure is to provide a small RNA capable of regulating cadmium content of crops, which can be applied in breeding of low-cadmium crops.

To achieve the above objective, the present disclosure adopts following technical solutions:

The present disclosure finds that miRNA (miR) 408 is largely induced by detecting an expression level of miRNA precursor of rice under a condition of heavy metal cadmium stress, and obtained a miR408 overexpressed rice material and a miR408 knockout rice material by constructing miR408 overexpression and CRISPR-cas9 expression vectors and transforming them to rice. The cadmium content in brown rice of the above materials planted in cadmium-contaminated soil is detected, and it is found that the cadmium content in the brown rice of the miR408 overexpressed rice decreases significantly, and that in the brown rice of the miR408 knockout rice increases significantly, indicating that the miR408 plays an important role in regulating the cadmium content of brown rice. There are no significant differences in agronomic traits (such as plant height, tiller number, spike length, and number of branches) exhibited by field planting between miR408 overexpressed rice and controls.

Therefore, the present disclosure provides use of miR408 with the nucleotide sequence set forth in SEQ ID No: 1 in regulation of cadmium accumulation in crops.

The use includes: using an overexpression technology to overexpress miRNA 408 in crops to obtain a transgenic plant with low cadmium accumulation in seeds.

Specifically, an MIR408 overexpressed vector is constructed and transformed into a recipient crop to obtain a crop material with significantly reduced cadmium content.

Further, the crops are monocotyledonous crops. In some embodiments, the crops may be rice and wheat. Using the MIR408 overexpressed vector constructed in the present disclosure to transform other crops can obtain crop materials with significantly reduced cadmium content, which can provide a material basis for cultivating a plurality of low-cadmium crops.

In some embodiments, miR408 precursor gene has the sequence set forth in SEQ ID No: 2.

The present disclosure further provides a breeding method for reducing a cadmium content in brown rice, including following steps:
step 1, cloning an MIR408 precursor gene with the nucleotide sequence set forth in SEQ ID No: 2 into an overexpression vector to obtain a recombinant vector; and
step 2, using an Agrobacterium-mediated transformation method to transform the recombinant vector into a recipient rice, cultivating and screening to obtain a rice plant with low cadmium accumulation in brown rice.

In some embodiments, the overexpression vector may be a pCUbi1390 vector.

In some embodiments, the recipient rice may be Oryza. Sativa L. spp. japonica.

The present disclosure has following beneficial effects:

The present disclosure constructs an overexpression vector of MIR408 precursor gene, and transforms it to a recipient rice to obtain a MIR408 gain-of-function transgenic rice, the MIR408 gain-of-function transgenic rice on the one hand can significantly inhibit cadmium accumulation in rice, and on the other hand, have no fundamentally effect on other agronomic traits. Therefore, the present disclosure can provide an important genetic material for breeding low-cadmium rice, and can be used for genetic transformation of other monocotyledonous crops, providing reference for other crops planted in cadmium-contaminated soil to reduce heavy metal hazards such as cadmium accumulation.

### BRIEF DESCRIPTION OF THE DRAWINGS

FIG. 1 illustrates cloning of MIR408 gene in Example 1;

FIG. 2 illustrates an MIR408 overexpressed cloning vector of pCUbi1390 in Example 1;

FIG. 3 illustrates MIR408 CRISPR-cas9 knockout cloning vectors of pYLCRISPR/Cas9Pubi-H and pYLsgRNA-OsU3/pYLsgRNA-OsU6a in Example 2;

FIG. 4 illustrates a genetic transformation process in Example 3, where □ to □ successively represent: callus induction, callus subculture, Agrobacterium infection, primary screening, resistant callus peeling, resistant callus subculture, resistant callus differentiation, rooting of regenerated shoots, and transplanting of regenerated shoots;

FIG. 5 illustrates a PCR identification result of hygromycin gene in an MIR408 overexpressed transgenic plant in Example 4;

FIG. 6 illustrates a semi-quantitative PCR identification result of an MIR408 overexpressed transgenic plant in Example 4;

FIG. 7 illustrates identification of mutation sites of an MIR408 CRISPR-cas9 knockout transgenic plant in Example 5;

FIG. 8 illustrates determination of Cd content in brown rice of an MIR408 overexpressed transgenic plant in Example 6;

FIG. 9 illustrates determination of Cd content in brown rice of an MIR408 knockout plant in Example 6;

FIG. 10 illustrates investigation results of agronomic traits of MIR408 overexpressed transgenic plants in Example 7;

FIG. 11 illustrates investigation results of agronomic traits of MIR408 knockout plants in Example 7.

### DETAILED DESCRIPTION OF THE EMBODIMENTS

The present disclosure will be further described below in conjunction with specific examples, but the present disclosure is not limited thereto.

### Example 1: Construction of MIR408 Overexpression Vector

PCR amplification primers were designed according to the sequence of MIR408. Using DNA of Oryza, Sativa L. spp. japonica material as a template, an MIR408 precursor gene was obtained by PCR amplification. After double digestion with KpnI and BamHI, the MIR408 precursor gene was ligated to a binary vector with UBI promoter.

1. Cloning of Rice MIR408 Gene

Using primers with restriction sites, forward primer CTCG̅G̅T̅A̅C̅C̅TGTACTCCAGCTTTGCTCTCC (set forth in SEQ ID No: 3) and reverse primer CTCG̅G̅A̅T̅C̅C̅CTAGCATGGCATGCCGAATGA (set forth in SEQ ID No: 4), the MIR408 gene was cloned from gDNA of Oryza. Sativa L. spp. japonica;

PCR program was: 1) 98° C. for 2 min; 2) 35 cycles of 98° C. for 10 s, 55° C. for 15 s, and 72° C. for 30 s; 3) 72° C. for 10 min.

The PCR system included:
5× PrimeSTAR Buffer (Mg²⁺ Plus), 10 μL;
dNTP Mixture (2.5 mM each), 4 μL;
forward primer (10 μM), 2 μL;
reverse primer (10 μM), 2 μL;
DNA template, 5 μL;
PrimeSTAR HS DNA Polymerase, 0.5 μL; and
making up to 50 μL with double distilled water.

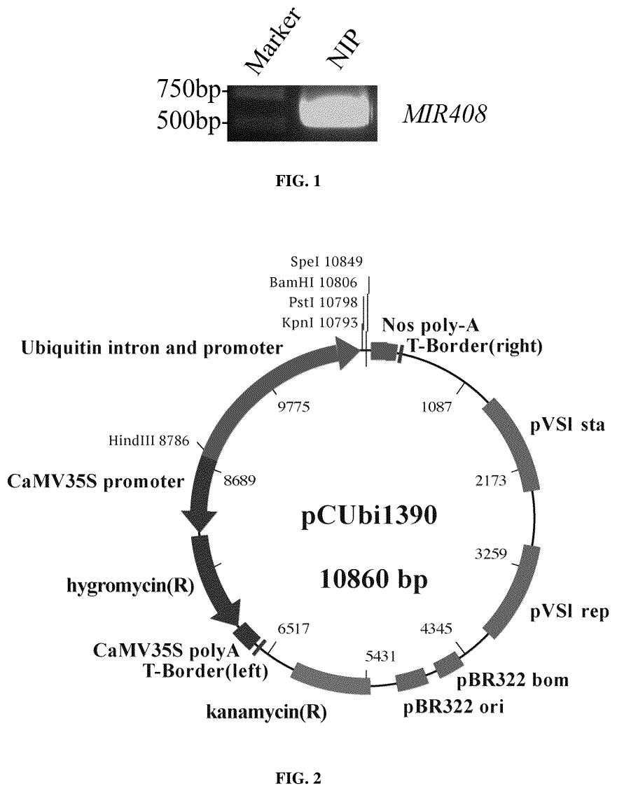

Cloning results were shown in FIG. 1, where the first column was DNA Marker, and the second column was a cloned product of MIR408 gene using Oryza. Sativa L. spp. Japonica as a template.

2. Construction of Rice MIR408 Overexpression Vector

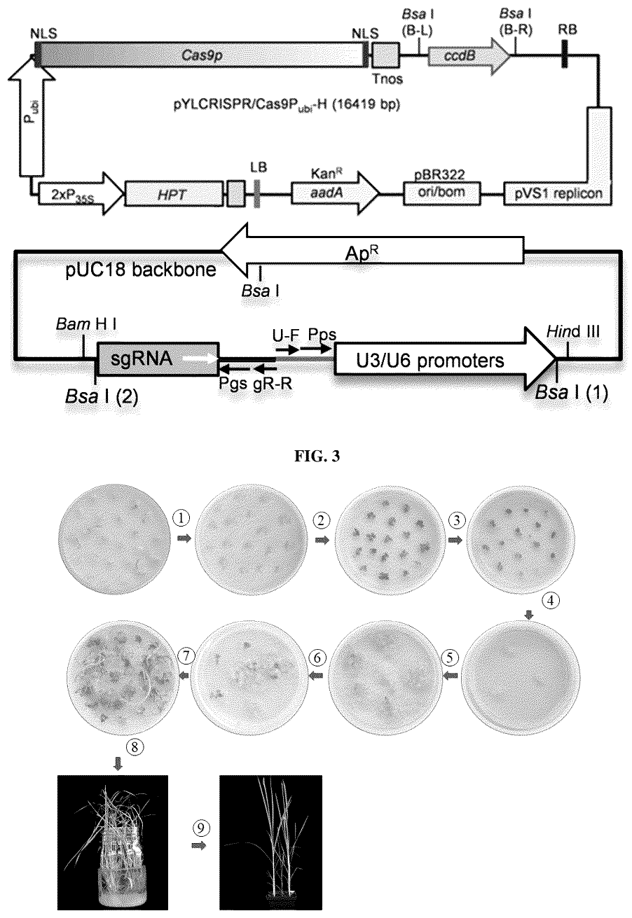

After gel extraction and purification, a PCR product in Example 1 (FIG. 1), together with the pCUbi1390 vector (FIG. 2), was double digested with KpnI and BamHI, respectively;
enzyme analysis program was: 37° C. for 5 min;
enzyme analysis system included:
10× FastDigest Buffer (Thermo Scientific), 2 μL;
Plasmid DNA, 1 μg or PCR product, 0.2 μg;
KpnI, 1 μL;
BamHI, 1 μL
making up to 20 μL with double distilled water.

After gel extraction and purification, all digested products were ligated:
ligation program was: 4° C. overnight;
ligation system included:
10×T4 DNA Ligase Buffer (Takara), 1 μL;
Plasmid DNA, 1 μL;
PCR product, 7 μL; and
T4 DNA Ligase, 1 μL.

The ligation product was transformed into Escherichia coli DH5α and multiplied therein. A positive clone was obtained by colony PCR and sequencing. A DNA sequence of the positive clone is set forth in SEQ ID NO: 2.

### Example 2: Construction of Rice MIR408 CRISPR-Cas9 Knockout Vector

Off-target prediction was conducted at a CRISPR Cas9 gene editing system website (http://skl.scau.edu.cn/) and finally the optimal target sequence was selected for primer design to construct PYLsgRNA-OsU3 and PYLsgRNA-OsU6a expression cassettes. Then a Cas9 vector and the two expression cassette fragments were assembled with BsaI and T4 by digesting-ligating.

The vector construction was mainly composed of three rounds of PCR amplification and digesting-ligating, and PCR program and system were the same as those in Example 1.

In a first round of PCR amplification: □ a pYLsgRNA-OsU3 vector (FIG. 3) was used as a template, U-F (set forth in SEQ ID No: 5) as a forward primer, and U3-miR408T target (set forth in SEQ ID No: 7)) as a reverse primer; □ a pYLsgRNA-OsU3 vector was used as a template, gRTU3-miR408 (set forth in SEQ ID No: 9) as a forward primer, and gR-R (set forth in SEQ ID No: 6) as a reverse primer; □ a pYLsgRNA-OsU6a vector was used as a template. U-F (set forth in SEQ ID No: 5) as a forward primer, U6a-miR408T target (set forth in SEQ ID No: 8) as a reverse primer; □ a pYLsgRNA-OsU6a vector was used as a template, gRTU6a-miR408 (set forth in SEQ ID No: 10) as a forward primer, and gR-R (set forth in SEQ ID No: 6) as a reverse primer for PCR amplification.

In a second round of PCR amplification: PCR products with using pYLsgRNA-OsU3 as a template in the first round of amplification were mixed for gel extraction; similarly, PCR products with using pYLsgRNA-OsU6a as a template in the first round of amplification were mixed for gel extraction to serve as templates of this round of PCR amplification, respectively; PCR amplification was conducted by using U-F (set forth in SEQ ID No: 5) as a forward primer and gR-R (set forth in SEQ ID No: 6) as a reverse primer.

In a third round of PCR amplification: the previous round of PCR products were recovered by gel extraction, respectively; PPS-GGL (set forth in SEQ ID No: 11) was used as a forward primer, and Pgs-GG2 (set forth in SEQ ID No: 12) was used as a reverse primer, respectively; PCR amplification was conducted by using PPS-GG2 (set forth in SEQ ID No: 13) as a forward primer and Pgs-GGR (set forth in SEQ ID No: 14) as a reverse primer, and PCR products were labeled as U3 and U6a after gel extraction.

Digesting-ligating: First, Digestion was conducted by using BsaI;
enzyme analysis program was: 37° C. for 15 min;
enzyme analysis system included:
cutsmart buffer (NEB), 1.5 μL;
U3, 3 μL;
U6a, 3 μL;
pYLCRISPR/Cas9Pubi-H, 1 μL;
BsaI, 1 μL;
making up to 15 μL with double distilled water.

After digestion, T4 Ligase and 10×T4 DNA Ligase Buffer were added to the above system for digesting-ligating. And a program of it was: 15 cycles of 37° C. for 5 min, 10° C. for 5 min, 20° C. for 5 min. A ligation product was transformed into E. coli DH5α, consistent with Example 1.

### Example 3: Genetic Transformation of MIR408 Gain-of-Function and Knockout Rice Materials

MIR408 gain-of-function and knockout binary vector constructed above was transformed into Agrobacterium tumefaciens AGL1; rice was transformed by an Agrobacterium-mediated transformation method to obtain hygromycin-resistant callus, and the callus was differentiated to obtain MIR408 gain-of-function and knockout transgenic rice materials.

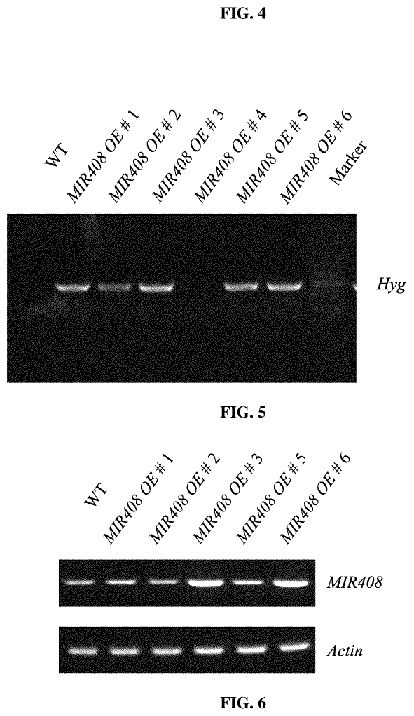

Steps of the Agrobacterium-mediated transformation method of rice were shown in FIG. 4:

1. Preparation of an Agrobacterium Bacteria Solution

A single colony of A. tumefaciens was picked on a clean bench, inoculated into 3-5 mL of Luria-Bertani (LB) liquid medium (supplemented with 20 mg/L rifampicin and 50 mg/L kanamycin), and shaken on a shaker at 28° C. and 250 r/min overnight until the bacterial solution became turbid and an OD₆₀₀ was about 1; 500 μL of the bacterial solution was diluted in 20 mL of LB liquid medium (supplemented with 20 mg/L rifampicin and 50 mg/L kanamycin), and kept shaking under the same conditions until the OD₆₀₀ was about 0.5. The obtained bacterial solution was centrifuged at room temperature and 4,000 r/min for 10 min, a supernatant was discarded, and bacteria were resuspended with 10 mM sterile MgSO₄ solution and centrifuged again, after the supernatant was discarded, the bacteria were still resuspended with the 10 mM sterile MgSO₄ solution and a final concentration of bacterial solution was adjusted to had an OD₆₀₀ of 0.2-0.3; after the concentration was adjusted, an obtained bacterial solution was centrifuged again, an obtained supernatant was discarded, and the bacteria were resuspended with an equal volume of Agrobacterium activation medium for infecting callus.

2. Induction and Subculture of Rice Callus

First, rice seeds were shelled, and seeds with full grains and no lesions were selected therefrom. The seeds were placed in a sterile tube in a clean bench, soaked in an appropriate amount of 75% alcohol for 1-2 min, and shaken continuously; subsequently, the alcohol was discarded, the seeds were washed with sterile water once, soaked and disinfected with 1% NaClO solution for 20-30 min, and shaken well; the seeds were rinsed with sterile water 3 to 4 times, and the seeds smelt NaClO-free were air-dried on sterile paper for later use; the sterilized seeds were placed evenly on a N6 medium, cultured in light at 26-28° C. for 7-10 days; after golden granular callus was induced, the callus was peeled onto a new N6 medium with tweezers. The callus that was subcultured for one week could be used for Agrobacterium infection.

3. Agrobacterium Transformation of Rice Callus and Co-Cultivation Process

A yellow and hard callus was selected, put into an activation medium with activated bacterial solution, shaken continuously for 5 min, and let stand for 30 min. The bacterial solution was discarded, the infected callus was placed on a sterile paper to drain the bacterial solution, and the drained callus was transferred to a solid co-cultivation medium and cultivated for 2-3 days at 28° C. in dark.

4. Screening and Subculture of Resistant Callus

The calli with a clean surface and no massive contamination by A. tumefaciens were transferred onto a primary screening medium for the first screening, and a screening was conducted once a week until the resistant callus grew (a new tender yellow callus grew around a brown callus). The resistant callus was peeled off to a new screening medium, and the callus peeled off from a single callus was a line. After another two weeks of screening, the line could be used for callus regeneration.

5. Regeneration of Resistant Callus

The screened callus was transferred onto a differentiation medium for continuous cultivation, and the callus was transferred to the rooting medium when the callus turned green and was differentiated into a plantlet.

### Example 4: Identification of MIR408 Overexpressed Transgenic Plants

Firstly, PCR cloning of hygromycin gene was conducted on an obtained MIR408 overexpressed TO transgenic plant. A genomic DNA of the transgenic plant was extracted as a template (using wild type as a negative control); PCR amplification was conducted by using Hyg-F (as shown in SEQ ID No: 15) as a forward primer and Hyg-R (as shown in SEQ ID No: 16) as a reverse primer. The PCR program and system were the same as those in Example 1. The results of PCR cloning of hygromycin gene were shown in FIG. 5: column 1 was wild type (WT) as a negative control, column 8 was DNA Marker, and columns 2 to 7 were six independent MIR408 overexpressed T0 transgenic lines obtained, respectively. Among them, MIR408 OE #4 failed to clone the hygromycin gene and was a false positive plant.

Then the screened positive transgenic plants were identified at a RNA level. RNAs were extracted from wild-type controls and transgenic plants and reverse-transcribed (reverse transcription reagent purchased from Promega), and MIR408 was relatively quantified by using OsActin as a reference gene to further determine whether the MIR408 was overexpressed.

A reverse transcription system was as follows:
RNA, 2 μg;
Oligo(dT)₁₅, 1 μL;
making up to 5 μL with DEPC-treated Water.

The above reaction system was mixed well, heated at 70° C. for 5 min, and immediately placed on ice for at least 5 min. Then a following mixture was added to the system:
GoScript™ 5×Reaction Buffer, 4 μL;
MgCl₂, 4 μL;
PCR Nucleotide Mix, 1 μL;
Recombinant RNasin Ribonuclease Inhibitor, 0.5 μL;
GoScript™ Reverse Transcriptase, 1 μL;
Nuclease-Free Water, 4.5 μL.

The reaction program was: 25° C. for 10 min, 42° C. for 1 h, and 70° C. for 15 min.

A product after reverse transcription was cDNA. First, the cDNA was leveled with internal control Actin, and then semi-quantitative PCR was performed to confirm whether the MIR408 gene was overexpressed. Steps of the process were as follows:

PCR amplification was conducted by using the reverse-transcribed cDNA as a template, q OsActin F (set forth in SEQ ID No: 17) as a forward primer, and q OsActin R (shown in SEQ ID No: 18) as a reverse primer. The reaction system and the reaction process were as shown in Example 1, but 22 cycles instead; an amount of templates was adjusted according to brightness of electrophoretic bands, so as to keep the brightness of the electrophoretic bands of the amplified products consistent.

After adjusting the amount of templates, semi-quantitative PCR was performed. Reverse transcribed cDNA was used as a template (the amount of templates was determined by an adjusted amount), q MIR408 F (as shown in SEQ ID No: 19) was used as a forward primer, and q MIR408 R (as shown in SEQ ID No: 20) was used as a reverse primer. The reaction system and the reaction process were as shown in Example 1, but 25 cycles were instead. Brightness of electrophoretic bands was used to judge whether overexpression was present. The semi-quantitative results were shown in FIG. 6. Under a condition that Actin was relatively consistent, expression levels of MIR408 OE #3 and MIR408 OE #6 in MR408 overexpression lines were significantly higher than those in wild type, so MIR408 OE #3 and MIR408 OE46 were true overexpression lines.

### Example 5: Identification of Mutation Sites in MIR408 Knockout Transgenic Plants

Firstly, PCR cloning of hygromycin gene was conducted on MIR408 knockout T0 transgenic plants obtained, which was consistent with Example 4.

Then mutation sites of the screened positive transgenic plants were identified. PCR amplification was conducted by using genomic DNA of a positive plant as a template, miR408-test-F (as shown in SEQ ID No: 21) as a forward primer and miR408-test-R (as shown in SEQ ID No: 22) as a reverse primer. The PCR program and the system were the same as those in Example 1.

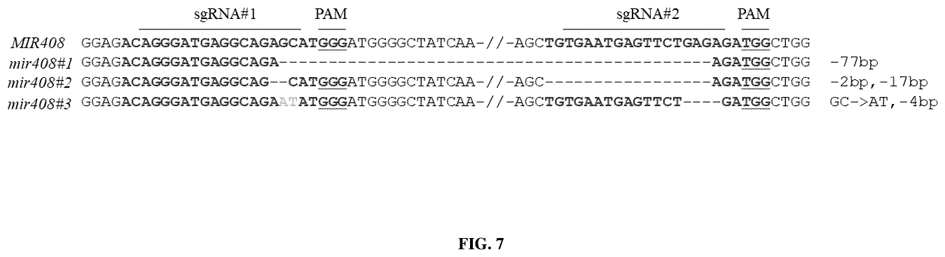

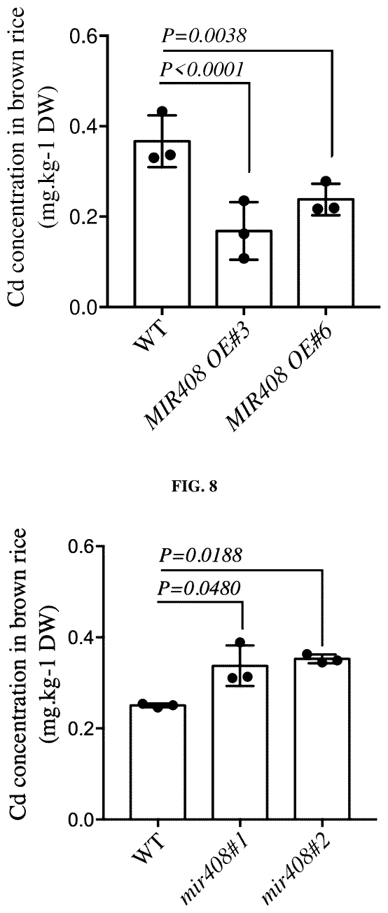

Finally, gel-cutting and sequencing of a PCR product was conducted, a sequencing peak pattern was analyzed, and mutations were counted. A statistical result was shown in FIG. 7.

### Example 6: Determination of Cd Content in Brown Rice of MIR408 Overexpressed and Knockout Transgenic Plants

A stable genetic MIR408 overexpressed transgenic plant, a knockout transgenic plant, and a wild-type rice plant was planted in a soil with Cd content of 2 mg/kg. Seeds of mature plants were harvested, dried, and shelled, and a Cd content of brown rice of the mature plants was determined.

A digestion process was as follows:
step 1, 0.2-0.4 g of brown rice was weighed into a digestion tube, supplemented with 3-5 mL of concentrated nitric acid, and placed in a fume hood overnight;
step 2, a temperature of the digestion furnace was adjusted to 90-100° C. and the digestion furnace was shaken slightly until brown rice shape disappeared into a fluid state; the temperature was properly raised to about 110° C., and the brown rice was heated for 2 h until it was completely liquid;
step 3, the temperature was raised to 140° C. again for acid discharge, and about 1 mL of a remaining liquid was retained in the digestion tube;
4. after cooling, a constant volume and diluting of the liquid was conducted, and the Cd content was determined.

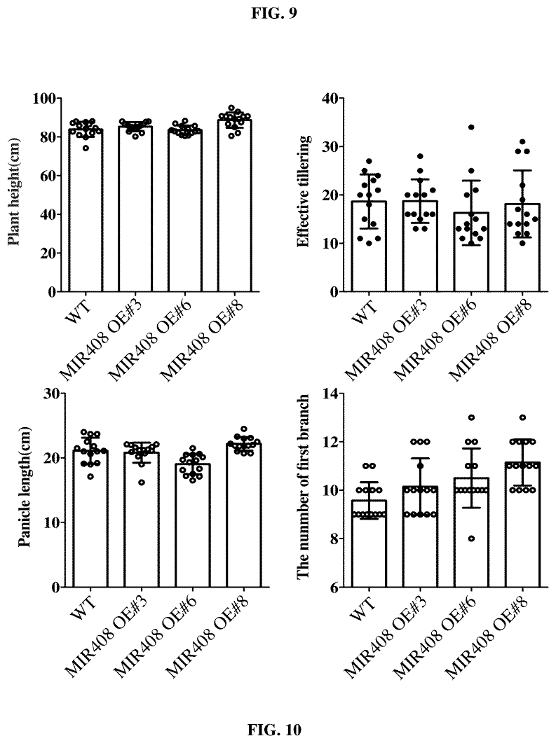

As shown in FIG. 8, a cadmium concentration in brown rice of MIR408 overexpressed transgenic plants planted in cadmium-contaminated soil was significantly lower than that in the control plants; as shown in FIG. 9, the cadmium concentration in brown rice of MIR408 knockout transgenic plants planted in cadmium-contaminated soil was significantly higher than that in control plants.

### Example 7: Results of an Investigation of Agronomic Traits of MIR408 Overexpressed and Knockout Transgenic Plants

A stable genetic MIR408 overexpressed transgenic plant, a knockout transgenic plant, and a wild-type rice plant were planted in the field at the same time, and their agronomic traits were individually investigated, including plant height, tiller number, spike length, branch number, and other important agronomic traits.

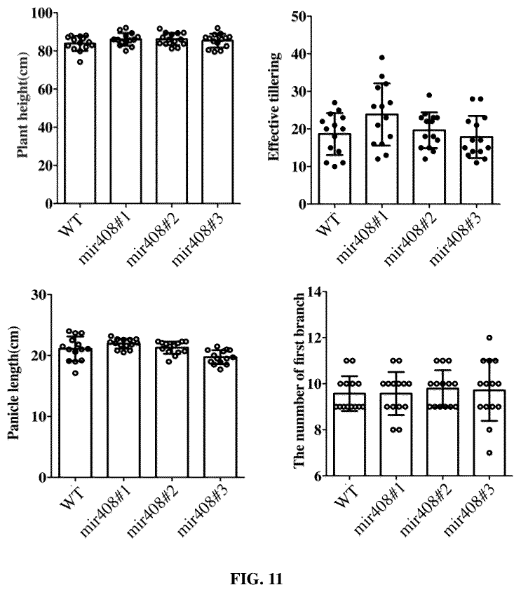

As shown in FIGS. 10 and 11, the agronomic traits of the MIR408 overexpressed and knockout transgenic plants were not significantly different from those of the wild type.

Although the present disclosure is described in detail in the foregoing examples, they are only a part of, not all of, the examples of the present disclosure. People can also obtain other examples according to the examples herein without creative efforts, and all of these examples shall fall within the claimed scope of the present disclosure.

1. A method of miR408 in regulation of cadmium accumulation in crops, wherein the nucleotide sequence of the miR408 is set forth in SEQ ID No: 1.

2. The method according to claim 1, wherein the regulation is inhibition.

3. The method according to claim 1, wherein the cadmium accumulation in crops is cadmium accumulation in crop seeds.

4. The method according to claim 1, wherein the use comprises: using overexpression technology to overexpress miR408 in crops to obtain a transgenic plant with low cadmium accumulation in seeds.

5. The method according to claim 4, wherein the sequence of a precursor gene of the miR408 is set forth in SEQ ID No: 2.

6. The method according to claim 1, wherein the crops are monocotyledonous crops.

7. The method according to claim 6, wherein the crops are rice and wheat.

8. A breeding method for reducing a cadmium content in brown rice, comprising following steps:
step 1, cloning an MIR408 precursor gene into an overexpression vector to obtain a recombinant vector, wherein the nucleotide sequence of the MIR408 precursor gene is set forth in SEQ ID No: 2; and
step 2, using an Agrobacterium-mediated transformation method to transform the recombinant vector into a recipient rice, cultivating and screening to obtain a rice plant with low cadmium accumulation in brown rice.

9. The breeding method according to claim 8, wherein the overexpression vector is a pCUbi1390 vector.

10. The breeding method according to claim 8, wherein the recipient rice is Oryza. Sativa L. spp. japonica.

11. A recombinant vector for reducing a cadmium content in brown rice, wherein the recombinant vector is obtained by cloning an MIR408 precursor gene into an overexpression vector, wherein the nucleotide sequence of a MIR408 precursor gene is set forth in SEQ ID No: 2, and the overexpression vector is pCUbi1390 vector.

12. An Agrobacterium strain comprising the recombinant vector according to claim 11.

13. The method according to claim 6, wherein the cadmium accumulation in crops is cadmium accumulation in crop seeds.

14. The method according to claim 6, wherein the method comprises: using overexpression technology to overexpress miR408 in crops to obtain a transgenic plant with low cadmium accumulation in seeds.


SEQUENCE LISTING (22 sequences)

SEQ ID NO: 1 - RNA, 21 nt, Artificial Sequence
  Nucleotide sequence of miR408
cugcacugccucuucccuggc

SEQ ID NO: 2 - DNA, 662 bp, Artificial Sequence
  DNA sequence of miR408 precursor gene
tgtactccagctttgctctccccttgtactgatggatgggcaaattggcaggctatgagatgtggtactagctagtactactgtactagcagtggaatggttcaaggcaaagacattgcgttgcgtgtgtatatatacatctcccatgtttcttgaatcttgacgatgatggcgttggcctaaccggatttgcagtgcatcaggtaagggaaaaaggatggttagatagagagaaggggagttctgtgattggagaggagaggagacagggatgaggcagagcatgggatggggctatcaacagatgtagattattccttgcacaagagatgatgatgagctgtgaatgagttctgagagatggctggtgttgttgttgctccctcccctgcactgcctcttccctggctcccctgcacacctctctctctctctctctctctctgtgtgttagtatattgtttttatgttttagtaattattttccttccataagtgatacggtatttgtagcatgtaggtagaaaaaaaaatgaaactagagtgatgtgacacttcaagggctgttgcaaattaacacattttaaaatggaccaagcgtactatttctcagcatgtggttctagctcaaaacatgcatttcattcggcatgccatgctag

SEQ ID NO: 3 - DNA, 30 bp, Artificial Sequence
  Forward primer of rice MIR408 gene
ctcggtacctgtactccagctttgctctcc

SEQ ID NO: 4 - DNA, 30 bp, Artificial Sequence
  Reverse primer of rice MIR408 gene
ctcggatccctagcatggcatgccgaatga

SEQ ID NO: 5 - DNA, 22 bp, Artificial Sequence
  Forward primer U-F
ctccgttttacctgtggaatcg

SEQ ID NO: 6 - DNA, 20 bp, Artificial Sequence
  Reverse primer gR-R
cggaggaaaattccatccac

SEQ ID NO: 7 - DNA, 37 bp, Artificial Sequence
  Reverse primer U3-miR408T target
tctctcagaactcattcacatgccacggatcatctgc

SEQ ID NO: 8 - DNA, 37 bp, Artificial Sequence
  Reverse primer U6a-miR408T target
tgctctgcctcatccctgtccggcagccaagccagca

SEQ ID NO: 9 - DNA, 37 bp, Artificial Sequence
  Forward primer gRTU3-miR408
tgtgaatgagttctgagagagttttagagctagaaat

SEQ ID NO: 10 - DNA, 37 bp, Artificial Sequence
  Forward primer gRTU6a-miR408
gacagggatgaggcagagcagttttagagctagaaat

SEQ ID NO: 11 - DNA, 38 bp, Artificial Sequence
  Forward primer PPS-GGL
ttcagaggtctctctcgcactggaatcggcagcaaagg

SEQ ID NO: 12 - DNA, 37 bp, Artificial Sequence
  Reverse primer Pgs-GG2
agcgtgggtctcgtcagggtccatccactccaagctc

SEQ ID NO: 13 - DNA, 38 bp, Artificial Sequence
  Forward primer PPS-GG2
ttcagaggtctctctgacactggaatcggcagcaaagg

SEQ ID NO: 14 - DNA, 37 bp, Artificial Sequence
  Reverse primer Pgs-GGR
agcgtgggtctcgaccgggtccatccactccaagctc

SEQ ID NO: 15 - DNA, 21 bp, Artificial Sequence
  Forward primer of Hyg-F
gcaagacctgcctgaaaccga

SEQ ID NO: 16 - DNA, 24 bp, Artificial Sequence
  Reverse primer Hyg-R
ctatcggcgagtacttctacacag

SEQ ID NO: 17 - DNA, 21 bp, Artificial Sequence
  Forward primer q OsActin F
tggcatctctcagcacattcc

SEQ ID NO: 18 - DNA, 20 bp, Artificial Sequence
  Reverse primer q OsActin R
tgcacaatggatgggtcaga

SEQ ID NO: 19 - DNA, 22 bp, Artificial Sequence
  Forward primer q MIR408 F
gattggagaggagaggagacag

SEQ ID NO: 20 - DNA, 21 bp, Artificial Sequence
  Reverse primer q MIR408 R
caacaacaacaccagccatct

SEQ ID NO: 21 - DNA, 18 bp, Artificial Sequence
  Forward primer miR408-test-F
atggttcaaggcaaagac

SEQ ID NO: 22 - DNA, 18 bp, Artificial Sequence
  Reverse primer miR408-test-R
tagtacgcttggtccatt